(19) United States
(12) Patent Application Publication
(10) Pub. No.: US 2021/0025871 A1
(43) Pub. Date: Jan. 28, 2021

(54) PURIFICATION PROCESS FOR CELLS

(71) Applicant: CSEM Centre Suisse d'Electronique et de Microtechnique SA - Recherche et Developpement, Neuchatel (CH)

(72) Inventor: Marc Zinggeler, Basel (CH)

(21) Appl. No.: 16/937,770

(22) Filed: Jul. 24, 2020

(30) Foreign Application Priority Data
Jul. 25, 2019 (CH) 00951/19

(51) Int. Cl. G01N 33/50; C12M 1/33; C12M 1/00; C12M 1/26

(52) U.S. Cl. CPC G01N 33/5091; C12M 33/14; C12M 45/05; C12M 45/02

(57) ABSTRACT

A process for isolating cells of interest from a sample comprising polluting cells. The process includes a filtration step through a membrane provided with pores and with linkers, and a crosslinking step allowing to bind the polluting cells to the membrane, leaving the cells of interest unbound.

### FIELD OF THE INVENTION

The present invention concerns a process for isolating one or more populations of cells from a sample in which they are present in a low concentration.

### DESCRIPTION OF RELATED ART

Identifying and isolating cells from a biological sample, such as a blood sample, is well-known for establishing a diagnostic test. Membranes are commonly used to this end, allowing to separate several populations of cells.

However, when the concentration of the cells to be identified is low compared to the other cells in the sample, the usual filtration processes can become inefficient.

In particular, the identification of circulating tumor cells (CTCs) and fetal cells can lack reliability. Due to their ultra-low concentration of only few cells per mL of blood, the cells must be isolated from a sufficiently large blood volume with high yield (i.e. close to 100%) and sufficient purity to enable molecular diagnostic procedures using existing analytical methods, such as immunocytochemistry (ICC), polymerase chain reaction (PCR) or sequencing. Other downstream methods, such as cell culturing, xenotransplantation or drug efficacy testing, may additionally require cells with unaffected biological properties.

Targeting molecules on the surface of the target cells does not always allow to collect the corresponding cells. Furthermore, the methods currently used for this purpose, such as workflows using magnetic beads, may strongly alter the biological properties, such as viability, of the target cells. The known negative selection strategies, including workflows using magnetic beads, comprise steps such as 1) incubating the blood with magnetic beads, which are functionalized with an antibody against a blood cell surface marker and pulling them out with a magnet (e.g. using EasySep™ from STEMCELL Technologies) or 2) crosslinking WBCs with RBCs and removing them by sedimentation (e.g. using RosetteSep™ from STEMCELL Technologies). These known methods are not suitable for large blood volumes. In addition, they do not allow to isolate the target cells with sufficient purity for straight-forward analysis. They have furthermore the risk of entrapping target cells (i.e. reduced yield).

Physical separation approaches, although more suitable to retain the biological integrity of the target cells, and for high-throughput processes, lack specificity. A high yield is usually obtained with a low purity or vice versa, a high purity induces a low yield.

Affinity membranes, comprising immobilized antibodies against target cell characteristic surface proteins are known, for example from US2013255361. However, this method is strongly dependant on the expression status of the cells with regard to the selected surface markers, resulting in the loss of some important cells. Furthermore, the targeted cells are bound to the membrane surface and their retrieval often results in a lack of viability or biological functionality.

There is therefore a room to improve the purification process to isolate cells being present in a biological sample in a low concentration, in particular circulating tumor cells and circulating fetal cells.

### BRIEF SUMMARY OF THE INVENTION

It is an object of the present invention to provide a purification process which allows to isolate cells of interest in a high yield and high purity. It is also an object of the present invention to increase both the yield and the purity of a purified population of cells.

It is an object of the present invention to provide a purification process for isolating cells which are present in a mixture of cells in a ratio lower than 10⁻⁷, 10⁻⁸, or 10⁻⁸, compared to the other cell populations in the sample.

It is an object of the present invention to provide a purification process adapted to retrieve unknown cells, or cells having unknown surface markers, or cells having no linkable surface markers, or cells which cannot be immobilized on a filtration membrane.

It is a further object of the present invention to provide a purification process which does not modify the functionalities of the targeted cells, and in particular, which does not modify the surface of the targeted cells or their surface markers.

It is an object of the present invention to provide a purification process adapted for the diagnosis of pathologies or infections related to unknown cells, or cells having unknown surface markers, or cells being present in a ratio lower than 10⁻⁷, 10⁻⁸, or 10⁻⁸, compared to the other cell populations in the sample.

It is an object of the present invention to provide a diagnostic method to identify circulating tumor cells or circulating fetal cells, in particular circulating fetal trophoblastic cells, or to improve the accuracy of the diagnostic methods used to identify these circulating cells.

It is a further aim of the present invention to provide a purification process which allows to concentrate the targeted cells.

It is also an object of the present invention to provide a purification process which is easily implemented and fast.

It is a further purpose of the present invention to provide a purification device adapted to the purification of cells which are present in a biological sample in a very low concentration, or which are not known or which cannot be immobilized on a filtration membrane.

According to the invention, these aims are achieved by means of the process and the device claimed in the independent claim 1 and further detailed through the dependent claims.

Above and below, the terms “fetal cells” designate any cell of a fetus, from its early embryo stage until its birth.

### BRIEF DESCRIPTION OF THE DRAWINGS

The invention will be better understood with the aid of the description of an embodiment given by way of example and illustrated by the following figures:

FIG. 1: schematic view of the cell purification process;

FIG. 2a: diagram of cell distribution after step S1 of the purification process;

FIG. 2b: diagram of cell distribution after step S5 of the purification process;

FIG. 3: example of crosslinking elements;

FIG. 4: schematic view of the pores of the filtration membrane;

FIG. 5a: Schematic view of a purification device according to an embodiment;

FIG. 5b, FIG. 5c: schematic views of the filtration device during the retrieving step S5.

### DETAILED DESCRIPTION OF POSSIBLE EMBODIMENTS OF THE INVENTION

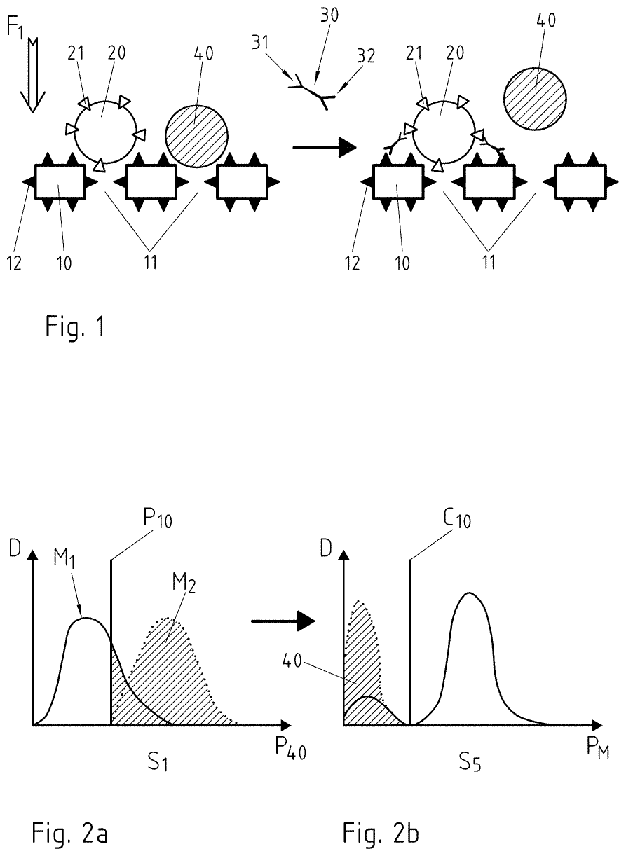

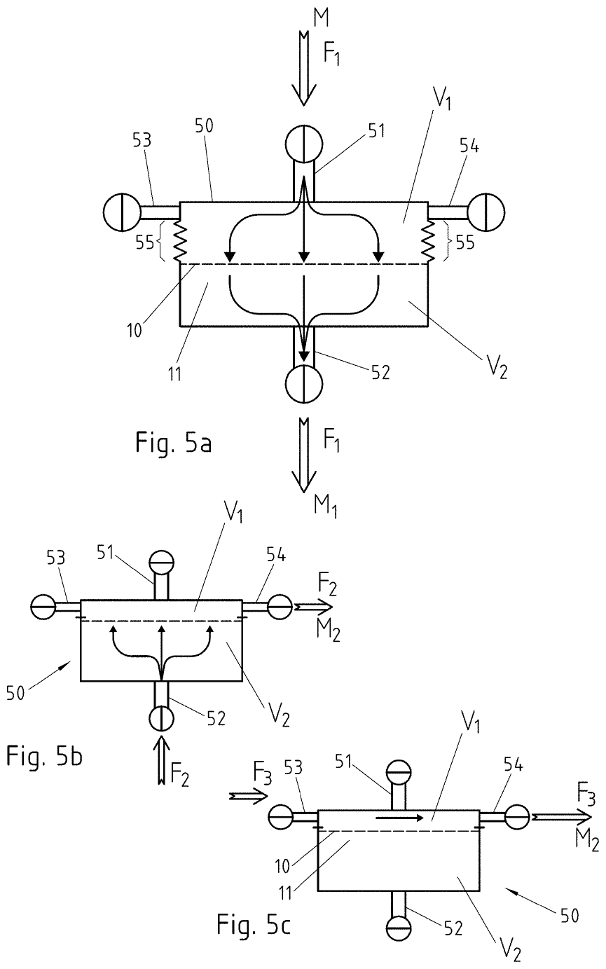

The present invention, as illustrated in FIGS. 1-5, provides a process for isolating cells of interest 40 from a sample mixture M (see FIG. 5a) comprising unwanted or polluting cells 20. The cells of interest 40 denote any cells which need to be identified, detected and/or analysed, for diagnostic or analytical purposes, as well as clusters thereof. A general process is illustrated in FIG. 1. Preferably, the cells of interest 40 should be kept alive in such a way they can be replicated in a cell culture. The cells of interests 40 should preferably also be kept functional. Their surface markers should thus be kept unmodified and substantially free of additional molecules which may disturb their biological functions. Alternatively or in addition, the cells of interest 40 may bear unknown surface markers in such a way that positive trapping using a specific linker is not possible or not suitable. Alternatively or in addition, the cells of interest 40 may be in such a low amount within the sample mixture M that they cannot be isolated with a sufficient yield or purity using known filtration processes. In particular, filtration processes based on physical properties of the cells may be too unspecific to isolate the cells of interest 40. For example, the cells of interest 40 may be circulating cells within the body such as circulating tumor cells, also known as CTCs, or circulating fetal cells or other circulating cells, like infectious cells.

The cells of interest 40 may be contained in one of any biological sample including blood sample, lymphatic fluid, urine sample, or cells arising from a biopsy sample or any other biological sample, and placed in a solution within a fluid such as a physiological fluid.

The polluting cells 20 comprise all the unwanted cells present within the biological sample mixture M.

The present process comprises a filtration step S1, consisting of filtering the sample mixture M of cells over a membrane 10 having pores 11. The sample mixture M comprises the cells of interest 40 among a quantity of other various cells in solution. The membrane 10 denotes any mesh or filtration device allowing to separate the cells of the sample mixture M based on their size or stiffness or both size and stiffness or other physical cell properties (e.g. nuclei size or stiffness). The cells of interest 40 have some physical parameters P40, which can be used to partially isolate them from the sample mixture M. The physical parameters P40 of the cells of interest 40 include the size of the cells of interest 40. The physical parameters P10 of the membrane 10 are preferably defined according to the physical parameters P40 of the cells of interest 40. The physical parameters P10 of the membrane 10 include the number, the size and the shape of the pores 11 (see FIG. 4). The physical parameters P10 of the membrane 10 allows to define a threshold to separate the retrieved mixture M2 and the waste mixture M1, during the filtration step S1, as shown in FIG. 2a. The cell distribution D within the waste mixture M1 and the retrieved mixture M2 may be similar, almost similar or different.

The membrane 10 may be a track-etched or a high-precision membrane. The membrane 10 may be for example selected from a polymer, a metal, a glass or a silicon based membrane like a silicon nitride material. It is to be understood that any suitable material may be used in relation to the purpose of the present process. The membrane 10 comprises pores 11. The pores 11 of the membrane 10 have a size comprised between 4 μm to 12 μm, preferably 5 μm to 8 μm. The pore size is preferably selected according to the size of the cells of interest 40. The pores 11 of the membrane 10 may have a cylindrical shape, or a conical shape or a slotted shape. A given membrane 10 may comprise a series of pores having a given shape and a series of pores having another shape. The membrane 10 having tapered pores 11, as shown in FIG. 4 is preferred due to the increased contact area between the membrane 10 and the cells of the mixture M, compared to a cylindrical pore 11. The density of pores 11 within the membrane 10 may be selected according to the needs of the filtration. For example, a density of few thousands of pores per cm² may be suitable. The density of pores 11 may be higher than this, up to 1 million per cm². The thickness of the membrane 10 may be comprised between 1 μm to 50 μm, preferably between 5 μm to 30 μm.

According to an embodiment, more than one membrane 10 may be used for the filtration step S1.

The membrane 10 is preferably coated with a cell repellent material in such a way to avoid unspecific adsorption on the membrane 10. In particular the membrane 10 may be coated with a hydrophilic uncharged polymer. The coating includes known hydrogels and polymers commonly used for this purpose. Preferably, the cell repellent coating is covalently bound to the membrane 10. The cell repellent coating is advantageously swelling when exposed to an aqueous solution. The linear swelling factor may be equal or higher than 1.5.

The membrane 10 is advantageously included or integrated to a filtration device 50 (see FIGS. 5a-c) comprising at least one inlet 51 to supply the mixture M of cells to the membrane 10 and at least one outlet to collect the eluted waste mixture M1, comprising the cells and other material which has passed through the pores 11 of the membrane 10. The filtration flow F1 goes from the inlet 51 toward the outlet 52 through the membrane 10.

The filtration step S1 is preferably performed at room temperature, namely around 20° C. A range of temperature comprised between 15° C. and 25° C. is also applicable. Alternatively, a colder temperature may be used. The filtration device 50 may be thermo-regulated in such a way that the temperature during the filtration process is controlled, either for the filtration step S1, either for another step of the filtration process.

The filtration step S1 may involve a pressure gradient or a pressure difference between the two opposite sides of the membrane 10. An overpressure may be supplied in the direction of the filtration flow F1 upstream the membrane 10. Alternatively or in addition, an underpressure may be applied downstream the membrane 10. According to a variant, a centrifugation force may be applied instead of an overpressure or an underpressure. The filtration device 50 may comprise one or more pressure sensors and a means for regulating the pressure. One or more means to induce a controlled hydrostatic pressure or pumps, such as peristaltic pumps may be connected or integrated to the filtration device 50. The pressure may be comprised between 20 to 200 mbar, preferably between 30 and 50 mbar. Lower pressures such as around 1 mbars or 10 mbars may also be applied, especially for diluted blood sample.

The time of the filtration step S1 is determined to avoid the degradation of the cells. It is preferably lower than one minute, typically comprised between 1 and 60 seconds. It is advantageous to have a membrane size or a density of pores or both membrane size and density of pores large enough to allow a fast filtration. A range of 200'000 to 400'000 pores per mL of the sample mixture M may be a suitable ratio. Other specific pore number may be considered depending on the type of sample mixture M to be dealt with.

Based on their physical properties, a part of the cells of the sample mixture M are eluted through the membrane 10, forming a waste mixture M1, and another part of the cells remain on the membrane 10, forming a retrieved mixture M2 of trapped cells. The physical parameters P10 of the membrane 10 and P40 of the cells of interest 40 contribute to define the cell distribution D during the filtration step S1. In addition, the conditions under which the filtration step S1 is performed can be modulated to increase the part of the cells of interest 40 within the retrieved mixture M2. In particular, the temperature and the pressure of the sample mixture M as well as the duration of filtration, may be monitored and controlled. The physical parameters, including the size and the shape of the pores 11 of the membrane 10, are determined in a way that the cells of interest 40 remain within the retrieved mixture M2.

The process of the present invention comprises a first washing step S2. The first washing step S2 is performed by supplying a suitable buffer in the direction of the filtration flow F1. The first washing step S2 may be understood as a front-washing step. Preferably, the physical conditions of the first washing step, such as the pressure gradient or pressure difference, and the temperature, are the same than those applied during the filtration step S1. Alternatively, one or more physical parameter may be different. The time of the first washing step S2 is also limited to one minute, preferably lower than 30 seconds, most preferably lower than 10 seconds. The suitable buffer can be supplied through the inlet 51 of the filtration device 50, already used for supplying the mixture M to the membrane 10. Alternatively, the suitable buffer can be supplied through another inlet 53. In a preferred arrangement, the same inlet is used for the filtration step S1 and the first washing step S2. Preferably, the filtration step S1 and the first washing step S2 are used in a single continuous process. An example of such a continuous process is described in Zinggeler et al. Sci. Rep. 2019. To this end, the biological sample to be filtered may be contained in a container (not shown) allowing containing the sample mixture M and the buffer used for the first washing step S2 in such a way that the buffer and the sample mixture M do not mix. Such a container may take the shape of a spiral tube allowing feeding the filtration device 50 with the sample mixture M and the buffer in a sequential and continuous way.

The first washing step S2 is preferably performed under a difference of pressure from one side of the membrane 10 to the other side. To this end, an overpressure may be applied on the side of the membrane 10 wherein the sample mixture M is supplied. Alternatively or in addition, an under-pressure may be applied downstream the membrane 10 to its surface opposite from the surface on which the sample mixture is supplied.

After the filtration step S1 and the first washing step S2 are performed, the retrieved mixture M2 of the trapped cells comprises the cells of interest 40 and several polluting cells 20. The type of the polluting cells 20 is preferably known. In particular one or more of the surface markers 21, characterising the polluting cells 20 are known and can be targeted with a specific complementary binding element 31. Alternatively, one or more subpopulations of the polluting cells 20 are known.

Preferably, the surface concentration PM of a given surface marker 21 is significantly higher for the polluting cells 20 than for the cells of interest 40. In other words, a surface marker 21 which is targeted to differentiate the polluting cells 20 and the cells of interest 40 is chosen in such a way that its surface concentration is significantly higher on the surface of the polluting cells 20, or a subpopulation of a polluting cells 20, than on the surface of the cells of interest 40. The surface concentration of a given surface marker 20 is significantly higher for a population of cells than for another one if it is at least twice, or ten times or hundred times higher than the surface concentration of the same surface marker 20 of another population of cells.

A binding element 31 denotes any small molecule, peptide, polypeptide, DNA oligomers or RNA oligomers, an antibody or a part of an antibody, or any other biochemical component able to specifically bind to a given surface marker 21 of the polluting cells 20. It is to be considered that the waste mixture M2 comprises several type of polluting cells 20, each of those having specific surface markers 21. Therefore, several specific binding element 31 may be used in such a way that most of the polluting cells 20 can be bond. All the binding elements 31 are selected in a way to not bind the targeted cells 40, or at least to preferably not bind the targeted cells 40.

The surface markers 21 related to the polluting cells 20 may be CD45, CD2, CD14, CD16, CD17, CD19, CD31, CD53, CD61, CD63, CD66b, CD69, CD81, CD84, glycophorin A, or any other suitable surface marker.

The binding elements 31 comprise or are combined with a linking element 32 able to cooperate with a linker 12 present on the surface of the membrane 10. The combination of the binding element 31 and the linking element 32 defines a crosslinking element 30 able to chemically bind the polluting cells 20 onto the surface of the membrane 10. The term chemically means that the polluting cells 20 are not only retained on the membrane 10 due to their physical properties but through chemical binding. The chemical binding includes weak interactions such as electrostatic or polar interactions and stronger interactions like covalent bonds. Preferably, the crosslinking elements 30 are defined to establish strong interactions between the linker 12 of the membrane 10 and the surface markers 21 of the polluting cells 20.

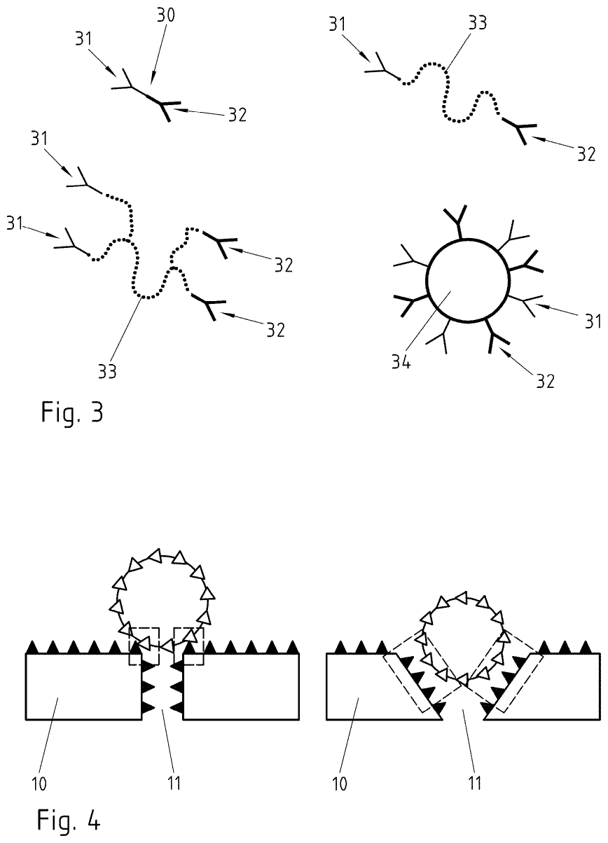

FIG. 3 shows various embodiments concerning the crosslinking element 30. The crosslinking element 30 may comprise a spacer 33 between the binding element 31 and the linking element 32. A spacer may be or may comprise any component able to increase the distance between the binding element 31 and the linking element 32 to facilitate the binding of the polluting cells 20. The spacer thus includes any molecular component, such as oligomers, organic polymers, micro-particles, or nano-particles 34. The spacer 33 may allow to combine one type of binding element 31 with a given linking element 32. Alternatively, the spacer 33 may allow to combine two different types of binding elements 21, 21′ with a given linking element 32 or several linking elements 32, 32′. The spacer 33 can be linear or branched. The binding elements 31 a spacer bears may be specific to the surface markers 21, 21′ of one type of polluting cells 20. Alternatively, the binding elements 31 of a given crosslinking element 30 may be specific to the surface markers 21, 21′ of different type of polluting cells 20. The spacer 33 may be selected among one or more of an organic polymer or a biopolymer, a peptide, a DNA or RNA oligomer, a micro-particle, and a nanoparticle.

The process of the present invention comprises a crosslinking step S3 allowing the polluting cells 20 to be strongly bond to the surface of the membrane 10. During this crosslinking step S3, the cells of interest 40 remain free, meaning unbound. The crosslinking step S3 comprises a step S3a of adding a crosslinking element 30 to the retrieved mixture M2 retained on the membrane 10. The crosslinking element 30 may be added as a solution within a buffer or a physiological fluid. The parameters such as the pH, the concentration and the type of the crosslinking element 30 may be adapted according to the specific needs. The crosslinking element 30 may be injected to the filtration device 50 through the inlet 51 or another inlet 53. Alternatively, a solution comprising the crosslinking element 30 may be placed in the same container as the one used for injecting the sample mixture M and the washing buffer in steps S1 and S2 into the filtration device 50, either in separated steps or in one combined steps. According to this one step procedure, the filtration step S1, the first washing step S2 and the step S3a of adding the crosslinking element 30 into the filtration device 50 may be performed in one continuous process.

Alternatively, the crosslinking element 30 may be stored as a dry reagent or a dry combination of reagents, such as a powder or a gel, or any other non-solubilised form, in a separate container, and added to the retrieved mixture M2 or to an intermediate reservoir comprising a suitable solvent. A mixing step can be included to facilitate the solubilisation of the crosslinking element 30 or its homogeneity.

The crosslinking step S3 comprises an incubation step S3b, allowing to perform the chemical binding of the crosslinking element 30 to the membrane and to the polluting cells 20. The incubating steps S3b may be performed according to well-known procedures. In particular, the binding of the binding element 31 and the corresponding surface marker 21 of the polluting cells 20 may be performed under conditions suitable for preserving the cells of the retrieved mixture M2 alive. The temperature, the pH, the duration and any other parameters are thus adaptable to the specific situation. For example, the temperature may be comprised between 18° C. and 22° C., preferably around 20° C., such as a room temperature. Alternatively, the temperature may be slightly above the room temperature to speed up the bonding process. Temperatures up to around 38° C. may be used. A temperature ranging from about 23° C. to about 37° C. is thus applicable to the present process. The incubation step S3b lasts preferably lower than an hour, most preferably lower than 30 minutes. The crosslinking conditions preferably allows to simultaneously bind the crosslinking element 30 to the linkers 12 of the membrane 10 and to the surface markers of the polluting cells 20.

The linker 12 on the membrane may be selected among streptavidin, biotin, a DNA or RNA oligomer, an antibody or an antibody segment, Ni-NTA complex, His-tag, an reactive agent such as an azide, an alkyne, or any other reactive chemical group.

The crosslinking step S3 may be followed by a second washing step S4 to elute the remaining reagents and unreacted elements. The second washing step S4 may be made under the same conditions as the first washing step S2. A suitable buffer or physiological fluid can be flowed in the direction of the filtration F1, following the front-washing procedure. The duration of the second step S3 is preferably less than one minute, such as 30 seconds or less than 10 seconds. The second washing step S4 is preferably performed under a difference of pressure from one side of the membrane 10 to the other side. To this end, an overpressure may be supplied in the direction of the filtration flow F1 upstream the membrane 10. Alternatively or in addition, an under-pressure may be applied downstream the membrane 10.

The second washing step S4 may alternatively be performed under different conditions than those of the first washing steps S2.

Following this second washing step S4, the polluting cells 20 present in the retrieved mixture M2 are bond to the membrane 10, whereas the cells of interest 40 remain unbound. In such a way to collect the cells of interest 40, a retrieving step S5 is performed to isolate the cells of interest 40 from the retrieved mixture M2. The retrieving step S5 is preferably performed by washing the membrane 10 in a direction different from the filtration direction F1.

A reverse flow F2 of an eluting fluid can be applied, according to a back-washing procedure. The eluting fluid is preferably a suitable buffer or a physiological fluid. A reversed difference of pressure compared to the one used during the filtration step S1 can be applied. The flow thus enters to filtration device 50 by the outlet 52 and goes through the membrane 10 in such a way that the unbound cells are removed from the membrane 10 and brought within the flow. The flow of eluting fluid can be directed through a specific outlet 54, allowing to collect the purified cells of interest 40. Alternatively, the flow of eluting fluid can enter the filtration device 50 through a specific inlet allowing it to cross the membrane in a direction opposite to the filtration flow F1.

Alternatively, the membrane 10 can be reversed, and the flow of eluting fluid can be inject in the direction of filtration F1. The unbound cells are thus retrieved from the membrane 10 through the outlet 52.

Alternatively, a laminar flow F3 of an eluting fluid can be flowed at the surface of the membrane 10 without crossing it, according to a shear-washing procedure. For example, the eluting fluid can be injected to the filtration device 50 by a specific inlet 53, on the side of the membrane containing the retrieved mixture M2, and collected through an output 54, arranged on the same side of the membrane as the inlet 53. The purified cells of interest 40 are thus collected.

The retrieving step S5 may include a sequential washing procedure comprising the back-washing and the shear-washing steps or a non-sequential combination thereof, wherein two different flows F2 and F3 are combined.

The retrieving step S5 can include a shaking step S5a wherein the membrane 10 is physically shaken in order to facilitate the separation of the cells of interest 40 from the membrane 10. The membrane 10 can be vibrated or shaken at a frequency which is not prejudicial to the cell integrity. The shaking of the membrane 10 can be performed with or without deformation of the membrane 10. Any suitable physical means of shaking the membrane can be used. A vibrating or shaking device may be incorporated within the filtration device 50 for this purpose. Preferably, the vibrating or shaking means exclude the use of ultrasounds, which may be prejudicial to the cell integrity. The vibration may be produced using a vortex generator for example or any equivalent known device. The shaking may be provided by the means of a translational periodical move either along one single direction or within a plan, using for example an eccentric rotational device. The shaking has low frequency compared to the vibrating. The vibrating or shaking device may allow the possibility to vary the frequency of move from a low shaking frequency, such as around 1 to 10 Hertz, to a higher vibrating frequency up to 100 or 1000 hertz, either progressively or using preselection ranges. The frequency may be higher than 1000 Hertz, provided that it remains lower that 10 000 Hertz. Alternatively, two distinct devices may be incorporated in the filtration device 50, a first one dedicated to high frequencies for vibrations, the other one being dedicated to lower frequencies adapted for a gentle shaking. The shaking step S5a results in the releasing of unbound cells from the membrane.

The yield of the purification process, and in particular of the retrieving step S5, depends on the surface concentration PM20 of the surface markers 20 compared to the surface concentration PM40 of the same surface marker on the surface of the cells of interest 40. Preferably, the concentration of the surface marker PM20 of the polluting cells 20 or a subpopulation of polluting cells 20, is higher than the concentration PM40 of the same surface marker on the cells of interest 40 by a factor of 2, or 10, preferably 100, 1000 or more. Preferably, the selected surface marker 20 is absent from the cells of interest 40. FIG. 2b shows the cell distribution D of the cells of interest 40 and the polluting cells 20 or a subpopulation of polluting cells 20, after the retrieving step S5. The chemical properties C30 of the crosslinking element 30 includes its selectivity toward the surface marker 20 of different cells of a mixture. The chemical properties C30 of the crosslinking element 30 allows to define a threshold to separate the cells of interest 40 and the polluting cells 20, or a subpopulation of a polluting cells 20, during the retrieving step S5.

The purification process of the present invention advantageously comprises a marker selection step SM, wherein the binding element 31 of the crosslinking element 30 is selected to specifically bind to a surface marker 21, the concentration of which is significantly higher on the surface of the polluting cells 20 than on the surface of the cells of interest 40. Several binding element 31 can be selected, each of those being selective of surface markers 21, 21′ of different subpopulations of polluting cells 20.

In order to concentrate the purified cells of interest 40 into a volume of fluid as small as possible, the filtration device 50 may comprise a dead-volume reducing means 55. This is preferred for many analytical or other procedures performed with the target cells. To this end, the filtration device 50 may comprise one or more flexible or adjustable walls, or part of walls, allowing to decrease the volume available on the membrane side comprising the retrieved mixture M2. Any other dead-volume reducing means can be used. For example, the membrane 10 can be arranged on a membrane holder which is movable in such a way to reduce the available volume on one side of the membrane 10. The filtration process can thus comprise a reducing step SR, wherein the dead-volume of the device 50 is reduced, either manually, either automatically, before initiating the retrieving step S5.

The filtration process comprises a collecting step S6, wherein the purified cells of interest 40 are collected. The collected cells of interest 40 are sufficiently pure and concentrated to be analysed, identified or cultured for multiplication. The collection of the purified cells of interest 40 is preferably done at a collecting outlet 54 different from the filtration outlet 52.

In case the collected cells of interest 40 are still polluted with one or more subtype of polluting cells 20, the purification process can be iterated once or more times, using either the same type of membrane 10, or a different type of membrane 10, having different physical parameters P10, and using a different crosslinking element 30, wherein the binding element 31 is selected to specifically bind to the remaining population of polluting cells 20. In particular, the present process optionally comprises the step of reiterating once or more times the retrieving step S5 or both filtration step S1 and retrieving step S5, using a membrane 10 having either the same physical properties P10 or different physical properties P10 and using a crosslinking element 30 having different chemical properties C30.

The present invention also comprises a filtration device 50, containing a filtration membrane 10, wherein the filtration membrane 10 defines a first volume V1 and a second volume V2 within the filtration device 50. The first volume V1 is in fluidic communication with at least one filtration inlet 51, adapted to inject a biological sample in contact to the membrane 10. The filtration inlet 51 may be connected to a container, wherein the biological sample, a washing fluid or any other fluids may be stored. The second volume V2 is in fluidic communication with at least one filtration output, adapted to collect the products eluted through the membrane 10.

One or more means to induce a controlled hydrostatic pressure or pumps, such as pumps, such as peristaltic pumps can be connected or integrated to the filtration device 50 in such a way to provide a pressure difference between the first V1 and the second V2 volumes. The pressure can be higher in the first volume V1 than in the second volume V2. The filtration device 50 can comprise a mean to reverse to pressure difference between the first volume V1 and the second volume V2, in a way that the pressure is lower in the first volume V1 than in the second volume V2.

The filtration device 50 advantageously comprises a dead-volume reducing means 55, in a way to reduce the first volume V1. The dead-volume reducing mean can comprise an adjusting wall or part of wall, allowing to reduce the internal volume of the filtration device 50, in particular to reduce the internal first volume V1 defined by the membrane 10.

The first volume V1 is advantageously in fluidic communication with a second inlet 53 and a second outlet 54, allowing to flow an eluting fluid along the membrane 10.

The filtration device 50 is conceived in such a way that it comprises at least two outlets, one of the two outlet being in fluidic connection with the first volume V1 and the other outlet being in fluidic connection with the second volume V2.

Optionally, the membrane 10 can be reversed in such a way to allow a back-washing procedure without inversing the pressure difference.

The filtration device 50 can in addition comprise a mixing chamber (not represented) allowing to introduce one or several components in a solid or semi-solid form into a solvent, and to solubilise such components into the solvent. Such a mixing chamber is preferably in fluidic connection with the first volume V1.

The filtration device 50 may in addition comprise one or more than one vibrating or shaking arrangement adapted to shake or vibrate the membrane 10.

Any one of the vibrating or shaking arrangement and pressure means can be independently activated, either manually or automatically. In a preferred arrangement, the filtration device 50 according to the present invention comprises a control unit (not represented) allowing to automatically pilot the filtration device 50 according to one or more preselected programs. The control unit allows in particular to induce and stop the steps of the purification process above-described according to a given program. The control unit may store several predefined programs. It may alternatively or in addition allow the user to set up new programs, corresponding to specific step arrangement of the present process, by means of one or more human machine interface such as a display, a keyboard, or any other known command devices.

The filtration device 50 may in addition comprise one or more sensors, such as pressure sensors, temperature sensors, optical sensors. It may be provided for example with a sensor adapted for counting particles in a flow of fluid.

The filtration device 50 may in addition comprise alert means such as sound generator or visual alarm means, so as to alert the user in case one or more parameters is sensed as being erroneous or different from a predefined value, or outside a predefined range of values. Over pressures or too high temperatures, or unexpected fluctuation of a parameter may thus be recorded and trigger an alarm message.

In another embodiment of the filtration device, the membrane 10 is contained in an insert which is compatible with centrifugation tubes. The process will then be run by a defined sequence of centrifugation steps which are executed manually or by a robot. The filtration pressure will be controlled by the rotation speed (g-force). To perform the retrieving step S5 the filter insert can be flipped around. To this end, the filtration device 50 may be provided with the necessary centrifugation means.

In another embodiment of the filtration device, the membrane 10 is contained in a tip which is compatible with pipettes or pipetting robots. The process will then be run by a defined sequence of pipetting steps which are executed manually or by a robot. To this end, the filtration device 50 may be provided with the necessary pipetting means.

The present invention further covers a diagnostic process comprising the purification process described above. In particular, the diagnostic process comprises a first step of retrieving a sample mixture M from the body. The sample mixture may be a sample of blood or a sample of urine or a sample of other body fluid or tissue. The diagnostic process further comprises a purification step by the means of the above-described purification process, in such a way that the targeted cells are isolated in a viable form. The diagnostic process may include a step of multiplying the isolated cells and analysing them. The diagnostic process is particularly adapted for early stage cancers or early stage metastatic phase identification, in particular because it is based on cells, such as CTCs cells, which are present at very low concentration. This furthermore allows to gain relevant information related to the therapy to be applied. The present diagnostic process is also advantageously applicable to prenatal diagnostic operations. In particular, fetal circulating cells may be involved in the present process to diagnose diseases before the birth. Circulating fetal trophoblastic (CFTCs) cells may be of particular interest for the purpose of the present non-invasive diagnostic test. Such a diagnostic test may be applied for the diagnosis of genetic abnormalities. For example, the Down Syndrome may be diagnosed according to the present diagnostic process.

The present diagnostic process may be applied in the same way as described here to cells other than CTCs cells or circulating fetal cells if these targeted cells are in very low concentration or have unknown surface markers. The present method is especially well suited for large sample volumes (large standard tube with 7.5 ml or 2 to 14 tubes of this volume) and low target cell concentrations (less than 100 cells/ml or less than 10 cells/ml or less than 1 cell/ml).

### Example 1: (Manual CTC-Isolation by Hydrostatic Pressure Filtration)

Used membrane: commercially available track-etched membrane disc with 25 mm diameter and pore size 8 μm (e.g. track-etched polycarbonate PCT8025100 from Sterlitech Corp.) or custom-made high-precision membrane disc with 25 mm diameter, 7.2 μm cylindrical pores arranged in hexagonal packaging and porosity of 23% and thickness of 15 μm made by hot-embossing in polycarbonate or by electroplating of nickel

The membrane is coated with hydrophilic polymer and functionalized with streptavidin linker by the process described in Zinggeler et al. Sci. Rep. 2019

The membrane is inserted in filter capsule (e.g. SX0002500 from Sigma-Aldrich). The Filter capsule is filled and washed with buffers (1. PBS-0.1% tween20, 2. PBS) using a syringe.

Undiluted EDTA blood sample (2 ml for track-etched membrane or 7.5 ml for high-precision membrane) is filtered at 34 mbar hydrostatic pressure and washed at the same pressure with PBS in one continuous step within 40 seconds using a setup requiring manual handling as described in Zinggeler et al. Sci. Rep. 2019.

Biotinylated anti-CD45 antibody solution (BAM1430, R&D Systems corp., diluted to 2 μg/ml in PBS) is injected into the capsule using a syringe and incubated at room temperature for 15 min.

Target cells are collected in a standard centrifugation tube by backwashing at 6 mbar hydrostatic pressure for 30 seconds.

### NUMBERS AND REFERENCE SIGNS OF THE FIGURES

10 Membrane
11 Pores
12 Linker
20 Polluting cells
21, 21′ Surface marker
30 Crosslinking element
31 Binding element
32, 32′ Linking element
33 Spacer
34 Nanoparticle/micro-particle
40 Cells of interest
50 Filtration device
51, 53 Inlet
52 outlet
54 Collecting outlet
55 Dead-volume reducing means
C30 Chemical properties of the crosslinking element 30
D Cell distribution
F1 Filtration flow
F2 Reversed flow
M Sample mixture
M1 Waste mixture
M2 Retrieved mixture
P10 Physical properties of the membrane 10
P40 Physical properties of the cells of interest 40
PM Surface concentration of a surface marker
PM20 Surface concentration of a given surface marker at the surface of the polluting cells 20
PM40 Surface concentration of a given surface marker at the surface of the cells of interest 40
S1 Filtration step
S2 First washing step
S3 Crosslinking step
S3b Incubation step
S4 Second washing step
S5 Retrieving step
S5a Shaking step
S6 Collecting step
SM Marker selection step
SR Reducing step
V1 First volume
V2 Second volume

1. A process for isolating cells of interest from a sample mixture comprising polluting cells, the process comprising a filtration step through a membrane, wherein the sample mixture is passing through a membrane following a filtration flow, wherein the membrane is provided with pores and with linkers, wherein the process comprises a crosslinking step allowing to bind the polluting cells to the membrane by the mean of a crosslinking element and to maintain the cells of interest unbound.

2. A process according to claim 1, wherein the filtration step provides a waste mixture eluted through the membrane and a retrieved mixture remaining within the membrane, and in that the size and the shape of the pores are determined in a way to maintain the cells of interest within the retrieved mixture.

3. Process according to claim 1, wherein the polluting cells comprise specific surface markers and that the crosslinking element comprises a binding element which specifically binds to one or more surface markers of the polluting cells.

4. Process according to claim 1, wherein the crosslinking step comprises a step of placing a crosslinking element in contact to the polluting cells and the membrane, and wherein the crosslinking step comprises an incubation step.

5. Process according to claim 1, wherein the crosslinking element comprises at least one binding element able to specifically bind to a surface marker of the polluting cells or a subpopulation of polluting cells, and at least one linking element adapted to cooperate with the linkers of the membrane.

6. Process according to claim 5, wherein the crosslinking element further comprises a spacer between the binding element and the linking element.

7. Process according to claim 6, wherein the spacer is selected among one or more of an organic polymer or a biopolymer, a peptide, an DNA or RNA oligomer, a micro-particle, a nanoparticle.

8. Process according to claim 5, wherein the binding element is selected from small molecule, peptide, polypeptide, DNA oligomers or RNA oligomers, an antibody or a part of an antibody.

9. Process according to claim 1 further comprising a retrieving step, allowing to collect the cells of interest.

10. Process according to claim 9, wherein the retrieving step comprises applying a flow of eluting fluid in a direction different from the filtration flow.

11. Process according to claim 10, wherein during said retrieving step (S5) a flow of an eluding fluid is applied through the membrane in a direction opposite to the filtration flow or a flow is applied along the membrane on its side containing the cells of interest.

12. Process according to claim 10, the retrieving step comprising a controlled shaking step facilitating the releasing of unbound cells from the membrane.

13. Process according to claim 9, optionally comprising the step of reiterating once or more times the retrieving step or both filtration step and retrieving step, using a membrane having either the same physical properties or different physical properties and using a crosslinking element having different chemical properties.

14. Process according to claim 1, wherein the cells of interest (40) are circulating tumor cells, fetal cells, or infectious cells.

15. Process according to claim 1, further comprising a marker selection step, wherein the binding element of the crosslinking element is selected to specifically bind to a surface marker, the surface concentration of which is significantly higher at the surface of the polluting cells or a subpopulation of polluting cells, compared to its surface concentration at the surface of the cells of interest.

16. A filtration device comprising a membrane which defines a first volume and a second volume, wherein the first volume is in fluidic connection with an inlet and the second volume is in fluidic connection with an outlet, characterized in that the filtration device further comprises a retrieving outlet in fluidic connection with the first volume.

17. Filtration device according to claim 16, comprising a dead-volume reducing mean allowing to reduce the first volume.

18. Filtration device according to claim 16, further comprising a shaking or vibrating arrangement to shake or vibrate the membrane at a frequency lower than 10 000 hertz.

19. Filtration device according to claim 16, comprising one or more of a pumping means, a centrifugation means, or a pipetting means.

20. A diagnostic process for identifying and detecting cancer, early phase cancer, genetic abnormalities such as Down syndrome characterized in that it comprises a purification process as described in claim 1, using a filtration device comprising a membrane which defines a first volume and a second volume, wherein the first volume is in fluidic connection with an inlet and the second volume is in fluidic connection with an outlet, characterized in that the filtration device further comprises a retrieving outlet in fluidic connection with the first volume.
